(19) United States
(12) Patent Application Publication
(10) Pub. No.: US 2014/0200500 A1
(43) Pub. Date: Jul. 17, 2014

(54) COMPRESSION BAND AND METHOD OF USE TO MITIGATE SWELLING AND DISCOMFORT AT A MESOTHERAPY TREATMENT AREA

(71) Applicant: Jeffrey A. Klein, San Juan Capistrano, CA (US)

(72) Inventor: Jeffrey A. Klein, San Juan Capistrano, CA (US)

(21) Appl. No.: 14/095,836

(22) Filed: Dec. 3, 2013

Related U.S. Application Data
(60) Provisional application No. 61/752,631, filed on Jan. 15, 2013.

(51) Int. Cl. A61F 13/12; A61F 13/62

(52) U.S. Cl. CPC A61F 13/12; A61F 13/622
USPC 602/43; 604/377; 604/385.01

(57) ABSTRACT

A device and method for mitigating swelling and discomfort in a mesotherapy treatment area is disclosed. A compression band is wrapped around the treated area multiple times a day for a period of about 1 hour for each session after undergoing mesotherapy treatment.

### CROSS-REFERENCE TO RELATED APPLICATIONS

This application is a non-provisional patent application which claims priority to U.S. Provisional Patent Application Ser. No. 61/752,631, filed on Jan. 15, 2013, the entire contents of which is expressly incorporated herein by reference.

### STATEMENT RE: FEDERALLY SPONSORED RESEARCH/DEVELOPMENT

Not Applicable

### BACKGROUND

The embodiments and methods disclosed herein relate to a device and method for mitigating swelling and discomfort after receiving mesotherapy treatment.

Mesotherapy treatment is a series of injections made directly into the site of pathology or problem area. The treatment involves multiple small volume (i.e., 0.2 ml) subcutaneous injections of medication (e.g., sodium deoxycholate) directly into the targeted fat deposit of the problem area. Deoxycholate acts as a biological detergent to lyse or dissolve and solubilize cell wall membranes and intercellular components. Immediate temporary side effects include discomfort and swelling or edema in the treated area.

Accordingly, there is a need in the art for a device methodology to mitigate the discomfort and swelling experienced by patients receiving mesotherapy treatment.

### BRIEF SUMMARY

The embodiments and methods disclosed herein address the needs discussed above, discussed below and those that are known in the art.

A compression band is disclosed herein which may be wrapped around a mesotherapy treatment area to apply pressure to the mesotherapy treatment area. The compression band may be worn multiple times a day for about 1 hour per session. This regimen mitigates or reduces the amount of swelling and discomfort experienced by patients who have received mesotherapy treatment. The compression band may be straight. Alternatively, the compression band may have a dome shaped structure to help keep the compression band properly positioned on the treatment area. For example, if the patient receives mesotherapy treatment at the submental neck-chin area, then the dome shaped structure is placed on the top of the patient's head and an elastic member is wrapped under the patient's submental neck-chin area to apply pressure to the mesotherapy treatment area (e.g., submental neck-chin area). The compression band may also have a means for receiving a fluid absorbent material to absorb and contain fluid excreted out of the mesotherapy treatment area.

More particularly, a mesotherapy compression band for mitigating swelling and discomfort after a patient has received a mesotherapy treatment is disclosed. The band may comprise a dome shaped structure, an elastic member and a fastening mechanism. The dome shaped structure may generally correspond to the curvature of a patient's head. The elastic member may define first and second distal end portions. The first end portion may be attached to the dome shaped structure. The fastening mechanism may comprise first and second parts which are removably attachable to each other. The first part of the fastening mechanism may be attached to the second distal end portion of the elastic member and the second part attached to the dome shaped structure.

The dome shaped structure may be fabricated from a washable fabric. The dome shaped structure may be about 7 inches in diameter when viewed from the top.

The fastening mechanism may be hooks and loops. The hooks may be the first part of the fastening mechanism. The loops may be the second part of the fastening mechanism. Alternatively, the first and second parts may be loops and hooks, respectively.

A linear distance between the first and second parts of the fastening mechanism when the elastic member is in the unstretched state may be about 22 inches. The elastic member may be stretched to about 27 inches to about 32 inches. The elastic member may have a width of about 3 inches.

The band may further comprise a liquid absorbent material disposed at a middle portion of the compression band. The liquid absorbent material may be a super absorbent polymer or cotton gauze material.

The band may further comprise a receiver and a liquid absorbent material. The receiver may be attached to a middle portion of the band. Also, the liquid absorbent material may be disposed within the receiver for absorbing fluid excreted out of the mesotherapy treatment area. The receiver may be removably attachable to the band.

In another aspect, a mesotherapy compression band for mitigating swelling and discomfort after a patient has undergone a mesotherapy treatment is disclosed. The band may comprise an elastic member, a fastening mechanism and a liquid absorbent material. The elastic member may define first and second distal end portions. The fastening mechanism may comprise first and second parts which are removably attachable to each other. The first part of the fastening mechanism may be attached to the first distal end portion of the elastic member. The second part of the fastening mechanism may be attached to the second distal end portion. The liquid absorbent material may be disposed at a middle portion of the elastic member for absorbing fluid excreted out of the mesotherapy treatment area.

The band may further comprise a receiver attached to the middle portion of the elastic member. The receiver may have an interior cavity for receiving the liquid absorbent material. The receiver may be removably attachable to the elastic member.

In another aspect, a method for mitigating swelling and discomfort at a mesotherapy treatment area of a patient is disclosed wherein the method may comprise the steps of: receiving mesotherapy treatment to an area of the patient; wrapping a compression band around the area; applying pressure to the mesotherapy treatment area; and sustaining pressure to the mesotherapy treatment area with the compression band for a period of about ½ hour or more.

In the method, the wrapping step may include the steps of disposing a middle portion of the elastic member at a submental chin-neck area of the patient; lifting opposed end portions of the elastic member upward until a desired pressure is applied to the submental chin-neck area; and attaching the opposed end portions of the elastic member to maintain the pressure to the submental chin-neck area handsfree.

The lifting step may include the step of stretching the elastic band from about 22 inches in an unstretched state to about 27 to about 32 inches in a stretched state.

The wrapping step may include the step of wrapping the compression band around the submental neck-chin area of the patient.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a perspective view of a first side of a compression band for mitigating swelling and discomfort after a mesotherapy treatment;

FIG. 2 is a perspective view of a second side of the compression band shown in FIG. 1;

FIG. 3 is a perspective view of the compression band shown in FIGS. 1 and 2 wrapped around a head of a patient for applying local pressure to the mesotherapy treatment area;

FIG. 4 is a perspective view of a second embodiment of the compression band illustrating a dome shaped structure to help maintain position of the compression band on the patient's head;

FIG. 5 is a perspective view of the compression band shown in FIG. 4 worn by the patient;

FIG. 6 is an enlarged perspective view of a middle portion of the compression band of either FIGS. 1-3 or FIGS. 4 and 5 illustrating a pressure adjustment mechanism for adjusting pressure applied to the mesotherapy treatment area, the pressure adjustment mechanism is used to absorbed excreted liquid; and

FIG. 7 is an enlarged perspective view of the middle portion of the compression band illustrating a second embodiment of the pressure adjustment mechanism.

### DETAILED DESCRIPTION

Referring now to the drawings, a compression band 10 is shown which applies pressure to a mesotherapy treatment area 12. The compression band 10 may be worn to apply pressure to the mesotherapy treatment area 12 multiple times a day for about 1 hour per session. To this end, the compression band 10 has first and second parts 14, 16 of a removably attachable fastener 18 (e.g., hooks and loops, snaps, buttons, and the like) attached to opposed distal end portions of an elongate elastic member 20. The first part 14 of the removably attachable fastener 18 may be attached to a first side 22 of the elongate elastic member 20. The second part 16 of the removably attachable fastener 18 may be attached to an opposed second side 24 of the elongate elastic member 20. After receiving mesotherapy treatment, the patient 25 wears the compression band 10 so that the compression band 10 applies pressure to the mesotherapy treatment area 10 multiple times a day for about 1 hour per session. By doing so, swelling and discomfort associated with the mesotherapy treatment is mitigated or resolved faster.

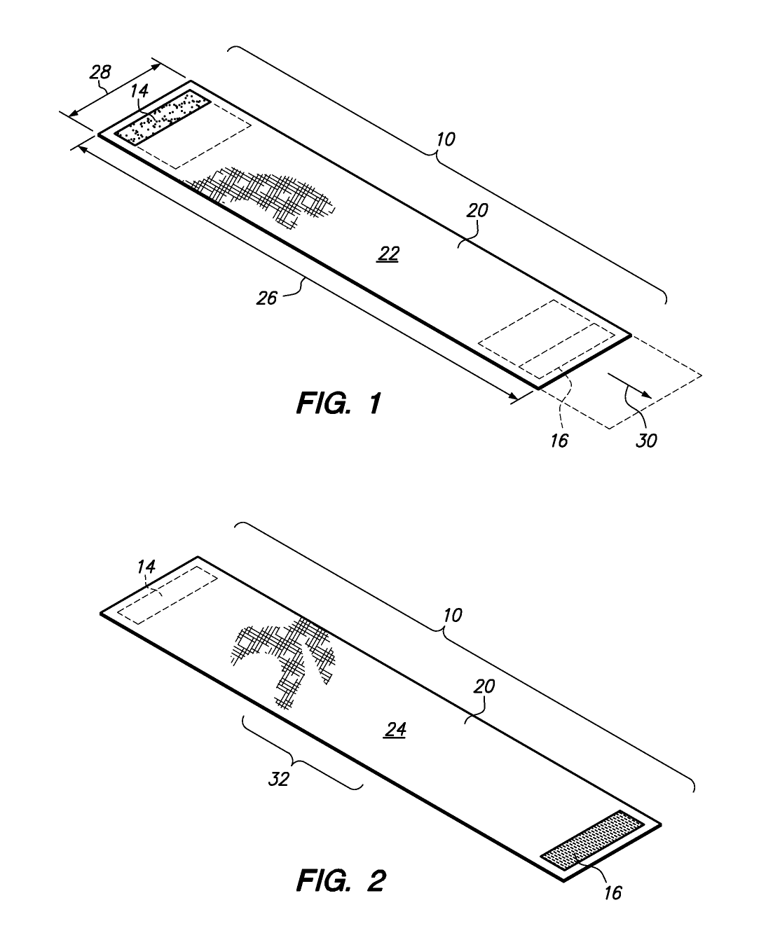

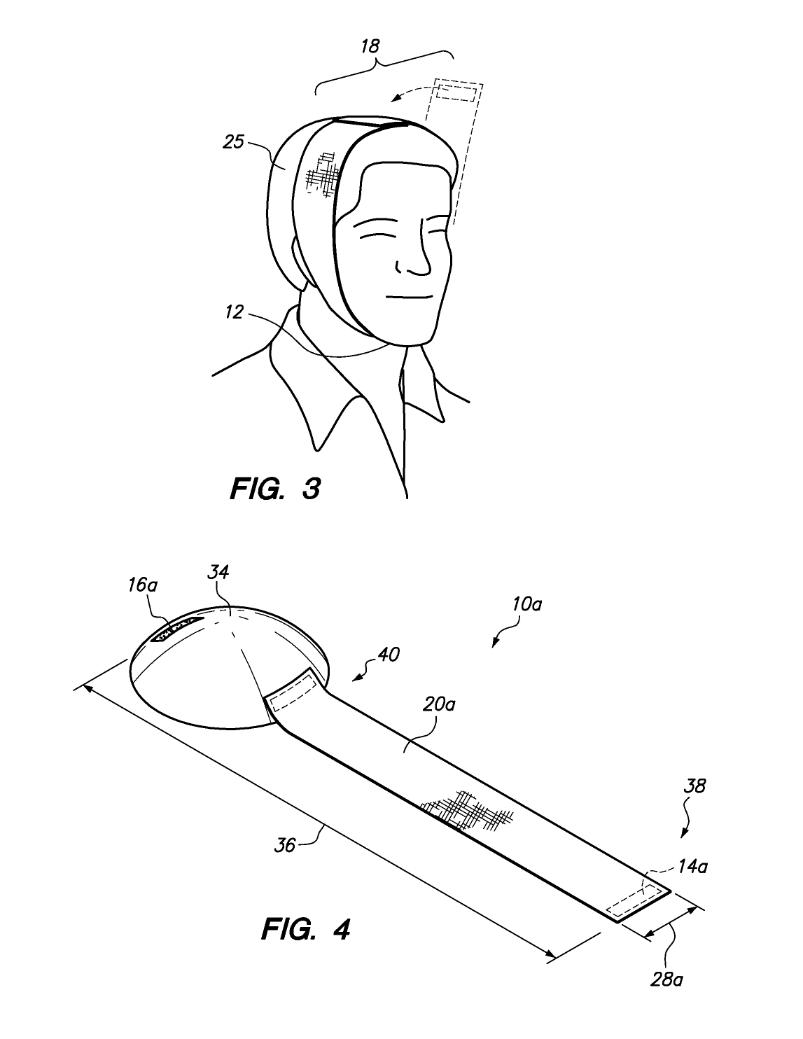

More particularly, referring now to FIG. 1, the elongate elastic member 20 may have a rectangular configuration defining a length 26 and a width 28. The elongate elastic member 20 may be fabricated from a stretchable and elastic fabric material. The elongate elastic member 20 may be elastic in the lengthwise direction. The length 26 of the elongate elastic member 20 may be sufficiently long so that the compression band 10 may be wrapped around a head of the patient 25 in the manner shown in FIG. 3. More particularly, the compression band 10 may be about 3 inches wide and 22 inches long. The compression band 10 may be extended to about 25 to 32 inches so that the compression band 10 may be wrapped under the patient's chin against the submental chin-neck area and over the top of the patient's head. The compression band 10 is stretched in the lengthwise direction as shown by arrow 30 and the first and second parts 14, 16 of the fastener 18 may be engaged to each other. When worn around the patient's head, the compression band 10 maintains pressure on the mesotherapy treatment area 12 in a hands free manner.

The compression band 10 shown in FIGS. 1 and 2 is bi directional in that the first side 22 or the second side 24 of the elongate elastic member 20 may contact the patient's skin. Although no friction material is embedded in the elastic member 20, it is contemplated that either the first side 22 of the second side 24 of the elongate elastic member 20 may be embedded with friction material. The side with the friction material contacts the patient's skin during use. The friction material mitigates slippage of the elastic member 20 once the band 10 is positioned on the patient's head. It is also contemplated that the friction material may be embedded on the opposed side of the elastic member 20 so that either side 22, 24 may contact the patient's skin during use and prevent slippage. It is also contemplated that the elastic member 20 may be fabricated from a material which has a high coefficient of friction. As used herein, a friction material or a high coefficient of friction is one that has a higher coefficient of friction than the coefficient of friction between a cotton fabric and the patient's skin.

In the method of using the compression band 10 to reducing swelling and discomfort after a mesotherapy treatment, the compression band 10 is placed on the patient's head after the patient undergoes mesotherapy treatment as shown in FIG. 3. The middle portion 32 is placed against the submental chin-neck area of the patient. Both hands of the patient 25 grasps the distal end portions of the compression band 10. The patient 25 pulls upward to stretch the compression band 10. In pulling upward, the compression band 10 places pressure on the submental chin-neck area of the patient 25. The patient 25 pulls up on the distal ends of the compression band 10 until the desired pressure is achieved. A first distal end portion of the compression band 10 is laid on top of the patient's head as shown in FIG. 3. A second distal end portion of the compression band 10 is lapped over the first distal end. The first and second parts 14, 16 of the fastener 18 are engaged to each other to hold the compression band 10 in place. The patient 25 wears the compression band 10 to place pressure on the mesotherapy treatment area preferably for a period of about 1 hour for two or more times a day until the swelling and discomfort has subsided. It is contemplated that the compression band 10 may be worn one or more times a day for at least ½ hour per session. The sessions may be spaced equally throughout the day. For example, the sessions may be at 8 am, 12noon, 4 pm and 8 pm. During sleep, the patient 25 may choose to take off the compression band 10. It is also contemplated that the patient 25 may sleep with the compression band 10 worn about the patient's head.

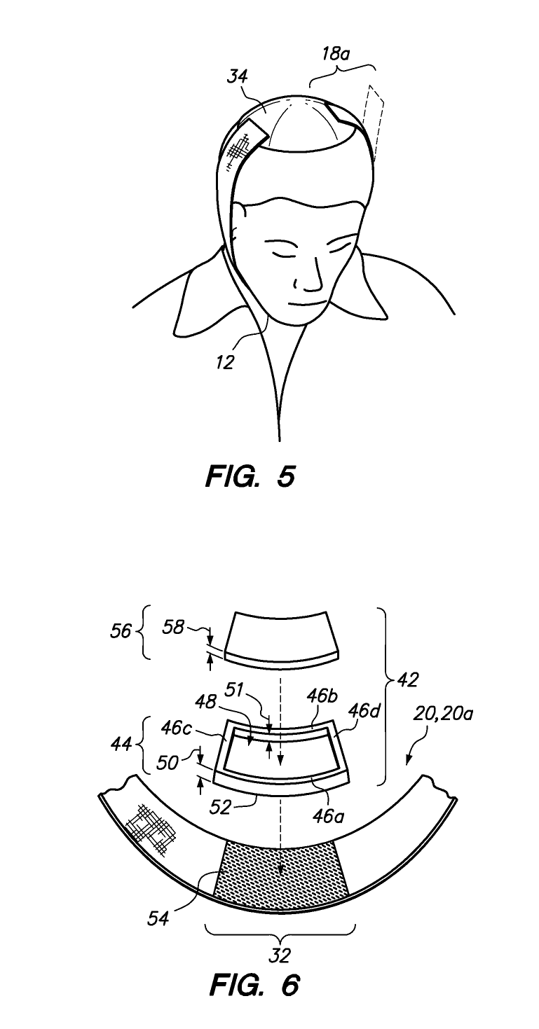

A second embodiment of the compression band 10a is shown in FIGS. 4 and 5. The compression band 10a may have an elongate elastic member 20a. The compression band 10a may additionally have a dome shaped structure 34. The dome shaped structure 34 may have a convex side which is shown in FIG. 4 and a concave side which engages or contacts the patient's head as shown in FIG. 5. The first and second parts 14a, 16a of the removably attachable fastener 18a may be attached to the distal end portion of the elastic member 20a and on an exterior surface of the dome shaped structure 34. The second part 16a attached on the dome shaped structure 34 may be attached adjacent to a perimeter of the dome shaped structure. The width 28a of the elastic member 20a may be about 3 inches. A distance 36 between the first and second parts 14a, 16a of the fastener 18a may be about 22 inches in the unstretched state. When stretched, the distance 36 may extend to between 27 to 32 inches so that the elastic member 20a may apply pressure to the submental chin neck area of the patient during recovery from the mesotherapy treatment.

More particularly, the dome shaped structure 34 may be about 5-7 inches in diameter when measured from the top. The dome shaped structure 34 may be fabricated from a fabric material or a semi rigid material that allows the dome shaped structure 34 to conform to the patient's head when worn and provide comfort so that the wearer does not experience any significant discomfort from the dome shaped structure 34. The concave surface engages the patient's head to prevents the compression band 10a from falling off of the patient's head during his/her session. The patient is able to move about without fear that the compression band 10a will slip off of his/her head.

The elongate elastic member 20a defines opposed distal end portions 38, 40. The distal end portion 38 is free and has the first part 14a of the fastener 18a attached to the underside of the elastic member 20a. The second distal end portion 40 is attached (e.g., stitching) to the dome shaped structure 34. The second part 16a of the fastener 18a is attached to the exterior surface near the perimeter of the dome shaped structure 34. The first and second parts 14a, 16a are aligned to a longitudinal axis of the elastic member 20a so that the elastic member 20a may be wrapped under the patient's chin as shown in FIG. 5 and the first and second part 14a, 16a of the fastener 18a attached to each other without twisting the elastic member 20a.

Referring now to FIG. 6, the middle portion 32 of elongate elastic member 20, 20a of the compression band 10, 10a may have a pressure adjustment mechanism 42. The pressure adjustment mechanism 42 may be permanently attached to the middle portion 32 of the elastic member 20, 20a. Alternatively, the pressure adjustment mechanism 42 may be removably attachable to the middle portion 32 of the elastic member 20, 20a of the compression band 10, 10a. The removeable attachment feature may be accomplished with hooks and looks, snaps or other mechanisms known in the art or developed in the future. The pressure adjustment mechanism 42 may have a shallow depth receiver 44. The receiver 44 may have front, back and side walls 46a, b, c, d which define an inner cavity 48. The receiver 44 may have also define a thickness 50 and inside depth 51. The receiver 44 may also be fabricated from a flexible and compressible material such as soft foam or rubber. This allows the receiver 44 to bend with the elastic member 20, 20a as the elastic member 20, 20a is wrapped around the patient's head. A lower side 52 of the receiver 44 may have a first part of a hooks and loops attachment system. The first or second side 22, 24 of the elastic member 20 at the middle portion 32 may have a second part 54 of the hooks and loops attachment system. The receiver 44 is removably attachable to the elastic member 20, 20a with the hooks and loops. Other removably attachable mechanism currently in existence or developed in the future may alternatively be utilized in lieu of the hooks and loops system.

A gauze pad 56 may be placed within the cavity 48. A thickness 58 of the gauze pad 56 may be greater than the depth 51 of the receiver 44 so that the gauze pad 56 and not the walls 46a-d places pressure on the submental chin-neck area of the patient 25. The mesotherapy treatment injects fluid into the subcutaneous fat tissue at the submental chin-neck area of the patient. In certain instances, the injected fluid leaks out. The gauze pad 56 is used to absorb the leaking fluid. The gauze pad 56 may be replaced during the session or after the session as needed. The gauze pad 56 may be manufactured from cotton. Alternatively, the gauze pad 56 may be a super absorbent polymer to absorb the leaking fluid. The super absorbent polymer may fit within the cavity 48 and also have a thickness 58 which is thicker than a thickness of the depth 51 so that the super absorbent polymer may apply pressure to the submental neck-chin area of the patient instead of the receiver.

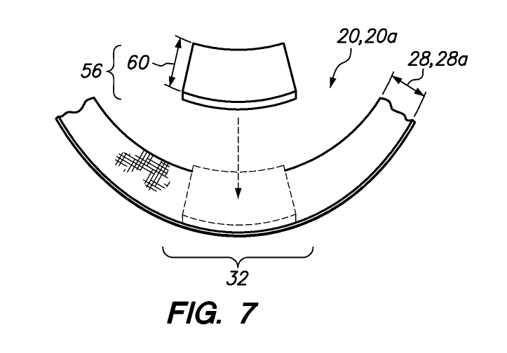

Referring now to FIG. 7, another embodiment is shown which illustrates a gauze pad 56 which is applied to the middle portion 32 of the elastic member 20, 20a. The gauze pad 56 may be positioned on the elastic member 20, 20a through friction. When the user wraps the compression band 10 around his/her head, the elastic member 20, 20a applies pressure to the submental neck-chin area through the gauze pad 56. It is this same pressure which holds the gauze pad 56 in place. The gauze pad 56 may have a width 60 which is less than, equal to or preferably greater than the width 28, 28a of the elastic member 20. Alternatively, an underside surface 62 of the gauze pad 56 may be removeably attached to the middle portion 32 of the elastic member 20, 20a with hooks and loops as discussed above. It is also contemplated that in lieu of the gauze pad 56, a super absorbent polymer may be used. It is also contemplated that the gauze pad 56 or the super absorbent polymer may be permanently attached to the middle portion 32 of the elastic member 20, 20a through stitching, adhesive, and the like. The gauze pad or the super absorbent polymer and the compression band 10, 10a may be washable to maintain sanitary conditions.

During use, the patient wraps the compression band 10, 10a about his/her head. The middle portion 32 of the elastic band 20, 20a is positioned under the patient's submental chin-neck area. The gauze pad 56 or the super absorbent polymer is placed in the receiver 44 (see FIG. 6) or placed directly on the elastic member 20, 20a (see FIG. 7). A thicker or thinner gauze pad 56 or super absorbent polymer is utilized depending on the desired pressure to be applied to the mesotherapy treatment area 12. With the gauze pad 56 or super absorbent polymer in position, the user disposes the middle portion 32 of the elastic member directly underneath the submental neck-chin area. Distal end portions 38, 40 of the elastic member 20, 20a are pulled upward until the desired pressure is applied to the patient's submental neck-chin area. The fastener 18, 18a is engaged to hold the compression band 10, 10a in place in a hands free manner. If the second embodiment of the compression band 10a is used, the user lifts the distal ends 38, 40 of the elastic member 20a until the dome shaped structure 34 is disposed on top of the patient's head. The fastener 18a is then engaged to hold the compression member 10a in place.

The above description of the compression band and the method of use of the compression band was explained in relation to mesotherapy treatment performed on the submental neck-chin area of a patient. However, mesotherapy may also be used to treat other areas such as the buttocks, knees, thighs or other fatty areas of a person's body. In these instances, the compression band may be modified so that it 10, 10a can be wrapped around the treated area. For example, when the mesotherapy treatment area is the thighs, the length of the compression band may be resized so that the compression band may apply pressure to the thighs. Moreover, the dome shaped structure may be modified to fit the curvature of the opposed side of the treatment area.

Moreover, the pressure adjustment mechanism 42 may be located at other areas of the elastic member 20, 20a. The pressure adjustment mechanism 42 may be located at either of the opposed distal end portions 38, 40 of the elastic member 20, 20a. Additionally it is contemplated that the first and second parts 14, 14a, 16, 16a may have varying lengths so that the mating parts amy engaged at different positions and vary the pressure applied by the band 10, 10a. More particularly, as shown in FIG. 1, the first part 14 may be lengthened as shown by the dash lines and/or the second part 16 may be lengthened as shown by the dash lines. In this manner, the first and second parts 14, 16 or 14a, 16a may be attached to each other to customize the stretched length of the elastic member 20 and apply the right amount of pressure to the mesotherapy treatment area.

The above description is given by way of example, and not limitation. Given the above disclosure, one skilled in the art could devise variations that are within the scope and spirit of the invention disclosed herein, including various ways of absorbing fluid from the mesotherapy treatment area of the patient. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A mesotherapy compression band for mitigating swelling and discomfort after a patient has undergone a mesotherapy treatment, the band comprising:
a dome shaped structure which generally corresponds to the curvature of a patient's head;
an elastic member defining first and second distal end portions, the first end portion being attached to the dome shaped structure;
a fastening mechanism comprising first and second parts which are removably attachable to each other, the first part of the fastening mechanism being attached to the second distal end portion of the elastic member and the second part attached to the dome shaped structure.

2. The band of claim 1 wherein the dome shaped structure is fabricated from a washable fabric.

3. The band of claim 1 wherein the fastening mechanism is hooks and loops, the hooks being the first part of the fastening mechanism and the loops being the second part of the fastening mechanism or vice versa.

4. The band of claim 2 wherein the dome shaped structure is about 7 inches in diameter.

5. The band of claim 1 wherein a linear distance between the first and second parts of the fastening mechanism when the elastic member is in the unstretched state is 22 inches.

6. The band of claim 5 wherein the elastic member is stretchable to about 27 inches to about 32 inches.

7. The band of claim 1 wherein the elastic member has a width of about 3 inches.

8. The band of claim 1 further comprising a liquid absorbent material disposed at a middle portion of the compression band.

9. The band of claim 8 wherein the liquid absorbent material is a super absorbent polymer or cotton gauze material.

10. The band of claim 1 further comprising:
a receiver attached to a middle portion of the elastic band; and
a liquid absorbent material disposable within the receiver for absorbing fluid excreted out of the mesotherapy treatment area.

11. The band of claim 1 wherein the receiver is removably attachable to the elastic band.

12. A mesotherapy compression band for mitigating swelling and discomfort after a patient has undergone a mesotherapy treatment, the band comprising:
an elastic member defining first and second distal end portions;
a fastening mechanism comprising first and second parts which are removably attachable to each other, the first part of the fastening mechanism being attached to the first distal end portion of the elastic member and the second part of the fastening mechanism being attached to the second distal end portion;
a liquid absorbent material disposed at a middle portion of the elastic member for absorbing fluid excreted out of the mesotherapy treatment area.

13. The band of claim 12 further comprising a receiver attached to the middle portion of the elastic member, the receiver having an interior cavity for receiving the liquid absorbent material.

14. The band of claim 13 wherein the receiver is removably attachable to the elastic member.

15. A method for mitigating swelling and discomfort at a mesotherapy treatment area of a patient, the method comprising the steps of:
undergoing mesotherapy treatment to an area of the patient;
wrapping a compression band around the area;
applying pressure to the mesotherapy treatment area;
sustaining pressure to the mesotherapy treatment area with the compression band for a period of about ½ hour or more; and
reapplying pressure two or more times per day.

16. The method of claim 15 wherein the wrapping step includes the steps of:
disposing a middle portion of the elastic member at a submental chin-neck area;
lifting opposed end portions of the elastic member upward until a desired pressure is applied to the submental chin-neck area;
attaching the opposed end portions of the elastic member to maintain the pressure to the submental chin-neck area handsfree.

17. The method of claim 16 wherein the lifting step includes the step of stretching the elastic band from about 22 inches in an unstretched state to about 27 to about 32 inches in a stretched state.

18. The method of claim 15 wherein the wrapping step includes the step of wrapping the compression band around the submental neck-chin area of the patient.
